(19) United States
(12) Patent Application Publication
(10) Pub. No.: US 2024/0402369 A1
(43) Pub. Date: Dec. 5, 2024

(54) QUALITY ASSURANCE PHANTOM FOR DIGITAL RADIOGRAPHY

(71) Applicant: Law Office of Jerry Joseph, PLC, Washington, DC (US)

(72) Inventors: John Isham, Nashville, TN (US); Clint Zeringue, Fulshear, TX (US)

(21) Appl. No.: 18/648,795

(22) Filed: Apr. 29, 2024

Related U.S. Application Data
(60) Provisional application No. 63/463,535, filed on May 2, 2023.

(51) Int. Cl. G01T 7/00; G01T 1/24; G01T 1/29

(52) U.S. Cl. CPC G01T 7/005; G01T 1/244; G01T 1/2992

(57) ABSTRACT

A quality assurance phantom configured for connection to a x-ray device including a housing member having an opening configured to receive a digital sensor, a frame member disposed within the housing member, the frame member configured to receive a phantom device, the phantom device comprising, a contrast layer configured to assess a contrast resolution, wherein the contrast layer is configured to provide two or more different contrast levels, and a spatial resolution test layer, wherein the spatial resolution test layer includes at least one trace disposed on a first surface of a substrate, and an attachment member configured to detachably secure the housing member to the x-ray device.

### BACKGROUND OF THE INVENTION

### 1. Field of the Invention

The present invention relates to dental imaging, and more specifically to a quality assurance phantom for digital radiography and a method of creating a quality assurance phantom.

### 2. Description of Related Art

Digital radiography has become a preferred method of medical imaging due to its ease of use and superior diagnostic capabilities. However, ongoing quality assurance is necessary to ensure that the digital imaging systems are producing accurate and consistent images. Additionally, many states require regular monitoring of dental radiographic equipment to ensure accurate diagnosis and that doses are kept as low as reasonably possible.

As such, contrast and spatial resolution phantoms have been previously developed to assist in performing quality assurance of these digital imaging systems. However, there are several disadvantages of conventional contrast and spatial resolution phantoms.

For instance, many contrast phantoms require careful alignment of the x-ray beam, source-to-object distance, and object-to-sensor distance to track digital image quality over time, which is time-consuming and cumbersome. Spatial resolution mechanisms are also problematic due to their high cost and time-consuming nature. Evaluating both contrast and resolution often requires overlaying a test pattern onto the contrast phantom, which adds an additional step of aligning the two separate phantoms.

Thus, there is a need for a quality assurance phantom that overcomes these issues by requiring less set up, being more efficient, and more cost-effective by integrating a contrast phantom, spatial resolution phantom, and digital sensor holder, into a single, self-aligning device that attaches directly to various x-ray devices.

### SUMMARY OF THE INVENTION

The present invention is an integrated quality assurance phantom for digital radiography that improves the speed and efficiency of digital imaging quality assurance tests. The present invention integrates a contrast phantom, spatial resolution mechanism, and digital sensor holder into a single device that mounts directly onto an x-ray device.

The present invention takes advantage of standard printed circuit board (PCB) manufacturing processes to create highly precise spatial resolution patterns using copper traces on a substrate or PCB. However, the present general inventive concept is not limited thereto. That is, in alternative embodiments, various other materials may be used for the traces and the traces may be formed in various complex design patterns, as desired. This novel and innovative process of the present invention of creating spatial resolution phantoms using traces on a substrate enables precise control over the thickness and width of the traces and allows for cost-effective production of quality assurance phantoms, at scale. Additionally, the method of assuring the quality of digital imaging systems using a quality assurance phantom according to the present invention reduces and/or eliminates the need for tedious and cumbersome alignment by integrating the contrast and spatial resolution layers into a single housing that is designed to attach directly to the x-ray machine. This improves the quality and speed of the quality assurance test and ensures consistent alignment over time and from person to person.

The device according to the present invention also enables quick insertion and detachment of digital sensors without requiring clamps, improving usability and the time required for testing. That is, the present general inventive concept includes a flexible holder designed and/or configured to dynamically secure and align various sized digital sensors inserted within the quality assurance phantom according to the present invention.

Certain of the foregoing and related aspects are readily attained according to the present general inventive concept by providing a quality assurance phantom configured for connection to an x-ray device including a housing member having an opening configured to receive a digital sensor or phosphorus plate, a frame member disposed within the housing member, the frame member configured to receive a phantom device, the phantom device including a contrast layer configured to assess a contrast resolution, wherein the contrast layer is configured to provide two or more different contrast levels and a spatial resolution test layer, wherein the spatial resolution test layer includes at least one trace disposed on a first surface of a substrate; and an attachment member configured to detachably secure the housing member to the x-ray device.

The quality assurance phantom may further include a flexible holder coupled to the frame member and configured to align a central axis of the digital sensor or phosphorus plate with a central axis of the frame member.

The flexible holder may further include a plurality of flexible members configured to secure and align the digital sensor or phosphorus plate with respect to the frame member.

The quality assurance phantom may further include a release mechanism movable from a first position to a second position and configured to eject the digital sensor or phosphorus plate from the opening of the housing member.

The attachment member may include an adjustable clamp member configured to align an x-ray transmission axis of the x-ray device with the phantom device.

The clamp member may include a curved contact pad configured to contact an exterior surface of the x-ray device.

The contrast layer may be formed from a single material having one or more sections, each section having a different thickness.

The material may include one of porcelain and aluminum.

The contrast layer may be formed from one or more different materials, the contrast layer having a uniform thickness.

The substrate may include a PCB material.

The one or more traces may be formed with different thicknesses.

The adjacent traces may be spaced apart at different widths.

The traces may be formed with a copper material.

The substrate may include a nonconductive material.

### BRIEF DESCRIPTIONS OF DRAWINGS

These and/or other aspects of the present general inventive concept will become apparent and more readily appreciated from the following description of the embodiments, taken in conjunction with the accompanying drawings of which:

FIG. 1 is a front perspective view of a quality assurance phantom for digital radiography according to an embodiment of the present general inventive concept;

FIG. 2 is bottom perspective view of the quality assurance phantom for digital radiography illustrated in FIG. 1;

FIG. 3 is an exploded assembly view of the quality assurance phantom illustrated in FIG. 1, with housing removed;

FIG. 4A is a front perspective assembly view of a conventional digital imaging sensor partially assembled into the quality assurance phantom illustrated in FIG. 1;

FIG. 4B is a top assembly view of a conventional digital imaging sensor completely assembled into the quality assurance phantom illustrated in FIG. 1, with housing removed;

FIG. 4C is a front perspective assembly view of a conventional digital imaging sensor ejected using the release mechanism;

FIG. 5A is an exploded assembly view of a quality assurance phantom according to an embodiment of the present general inventive concept and a conventional dental x-ray device;

FIG. 5B is an assembly view of the quality assurance phantom and the conventional dental x-ray device illustrated in FIG. 5A;

FIG. 6A is a schematic illustration of an alignment of a contrast layer, a spatial resolution layer, and alignment frame of the quality assurance phantom and a digital sensor with an axis of a conventional dental x-ray device;

FIG. 6B is an enlarged schematic illustration of FIG. 6A, with the alignment frame removed;

FIG. 7A is an exploded assembly view of a quality assurance phantom according to an embodiment of the present general inventive concept and a conventional hand-held dental x-ray device;

FIG. 7B is an assembly view of the quality assurance phantom and the conventional hand-held dental x-ray device illustrated in FIG. 7A;

FIG. 8 is a flowchart of a method of assuring the quality of digital imaging system according to an embodiment of the present inventive concept;

FIG. 9 is a flowchart of a method of creating a quality assurance phantom for an x-ray device according to an embodiment of the present inventive concept;

FIG. 10 is a front perspective view of the spatial resolution test pattern layer of the quality assurance phantom illustrated in FIG. 3;

FIG. 11A is a top plan view of the spatial resolution test pattern layer illustrated in FIG. 10;

FIG. 11B is a cross-sectional view of the spatial resolution test pattern layer in FIG. 11A, along the A-A line;

FIG. 11C is an enlarged schematic view of the spatial resolution test pattern layer illustrated in FIG. 11B;

FIG. 12 is a front perspective view of a contrast layer of the quality assurance phantom illustrated in FIG. 3;

FIG. 13 is a side view of the contrast layer of the quality assurance phantom illustrated in FIG. 12;

FIG. 14A is a front perspective assembly view of a contrast layer coupled to a spatial resolution test pattern layer with hidden lines visible illustrating traces disposed on the substrate, according to an embodiment of the present invention;

FIG. 14B is a top plan view of the contrast layer coupled to the spatial resolution test pattern layer illustrated in FIG. 14A;

FIG. 14C is a side view of the contrast layer coupled to the spatial resolution test pattern layer illustrated in FIG. 14A; and

FIG. 15 is a front perspective view of a quality assurance phantom for digital radiography according to an alternative embodiment of the present general inventive concept.

### DETAILED DESCRIPTION OF INVENTION

The definitions of selected terms disclosed herein are examples that fall within the scope of the term and are not intended to be limiting. The term substantially may be used as a modifier for a geometric relationship between elements or for the shape of a component or element of the present general inventive concept. As should be noted, the term substantially is not limited to a specific variation and may cover any variation that would be understood by one of ordinary skill in the art to be an acceptable variation. For example, in one embodiment the term substantially may refer to a variation of less than 25% of the dimension of the component or element. In another embodiment, the term substantially may refer to a variation of less than 5% of the dimension of the component or element.

A “dental x-ray device” refers to a medical device used to capture images of a patient's teeth, jaw, and surrounding tissues through the use of x-ray technology. The dental x-ray device typically consists of an x-ray tube that emits a small, controlled amount of radiation, and a digital or film sensor (i.e., digital sensor) that captures the x-ray image.

Dental x-ray devices are used by dentists and dental specialists to diagnose and monitor a variety of dental conditions, including cavities, gum disease, impacted teeth, and jaw problems. The images produced by the dental x-ray devices can help the dentist to identify potential issues before they become more serious and can aid in the development of effective treatment plans. Dental x-ray devices are subject to strict safety standards to ensure that patients are not exposed to unnecessary radiation, and to protect the operator from any harmful effects of ionizing radiation.

The dental x-ray device may also include a beam limiting device to restrict a size and shape of the x-ray beam that is emitted by the dental x-ray device. The beam limiting device is typically placed in front of an x-ray tube of the dental x-ray device and can be adjusted to limit an area of exposure to a specific region of a patient's mouth, thus reducing an amount of scattered radiation and increasing an accuracy of the x-ray image. The beam limiting device is an essential safety feature in dental x-ray devices, as it helps to minimize the patient's exposure to unnecessary radiation and ensures the highest quality images are obtained.

Contrast and spatial resolution phantoms are used in dental digital radiography quality assurance. The term “contrast phantom” refers to a device designed to evaluate the performance of dental x-ray imaging systems. The contrast phantom typically consists of a series of test objects with varying thicknesses and densities which mimic the anatomical features of the human teeth and surrounding tissues. For example, some contrast phantoms consist of a material similar to aluminum (Al) arranged in a staircase or stepped design, wherein each step has a different thickness. Each step would mimic a different part of the human body.

During a quality assurance evaluation, the contrast phantom is exposed to x-rays, and the resulting images are analyzed for various quality assurance parameters. The resulting images are then evaluated for accuracy, contrast, and resolution, and any deviations from the expected results can indicate problems with the imaging system. The use of a contrast phantom in dental digital radiography quality assurance is an important tool for ensuring accurate and reliable diagnostic imaging in dental clinics and hospitals.

The term “spatial resolution phantom” refers to a specialized tool designed to evaluate the ability of a dental x-ray imaging system to capture fine details and distinguish between adjacent structures. The spatial resolution phantom typically consists of a pattern of small, high-contrast elements arranged in a grid or line pattern.

During a quality assurance evaluation, the spatial resolution phantom is positioned in the x-ray beam, and the resulting image is analyzed for the ability to resolve and distinguish individual elements.

The spatial resolution phantom is a tool for quality assurance in dental radiography, as it can help identify potential problems with image resolution and provide a measure of the imaging system's overall performance.

The term “traces” refer to the conductive pathways that are formed on a substrate, such as a printed circuit board (PCB), to connect electronic components in a circuit. Traces are typically made by applying a layer of conductive material, such as copper, onto the substrate, and then selectively removing the material in certain areas to create the desired pattern of pathways.

Conventional manufacturing techniques for manufacturing PCBs can achieve a very high precision in the placement and routing of traces. For example, it is possible to create traces that are only a few microns in width, with precise spacing and alignment between adjacent traces.

The precision of traces is typically measured in terms of their resolution, which refers to the smallest width or spacing that can be achieved between adjacent traces. The resolution of traces can vary depending on the specific manufacturing process used, but in general, resolutions in the range of 10-20 microns are considered achievable using modern techniques.

Reference will now be made in detail to the exemplary embodiments of the present general inventive concept, examples of which are illustrated in the accompanying drawings, wherein like reference numerals refer to the like elements throughout. The exemplary embodiments are described below in order to explain the present general inventive concept. The following description presents a simplified summary of one or more aspects of the present general inventive concept. However, the present general inventive concept is not limited thereto.

The present general inventive concept comprises a quality assurance phantom for digital radiography that is designed to assess contrast, spatial resolution, artifacts, and noise of a digital imaging system over time. The present general inventive concept further comprises a method of assuring the quality of digital imaging of various types of x-ray systems including intraoral x-ray devices, handheld x-ray devices, stationary x-ray devices, and veterinary x-ray devices. The present general inventive concept further comprises a method of creating an integrated quality assurance phantom for an x-ray device.

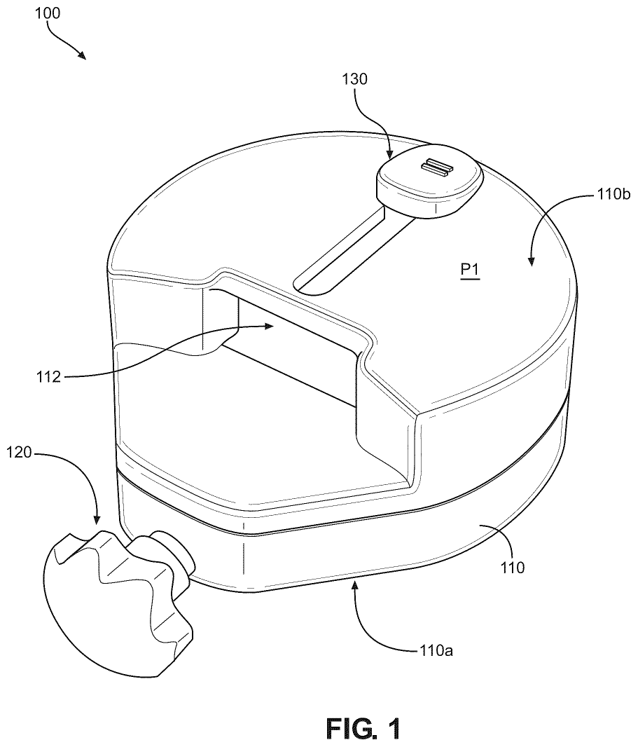

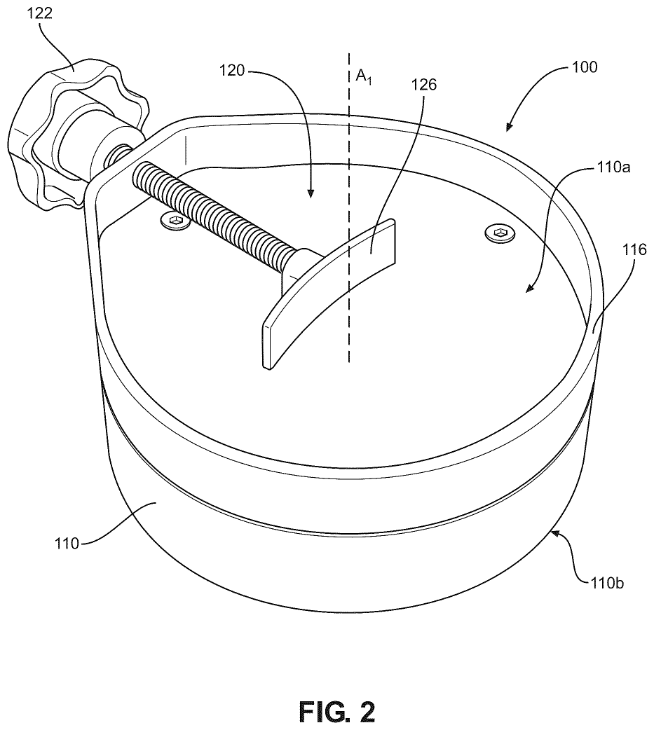

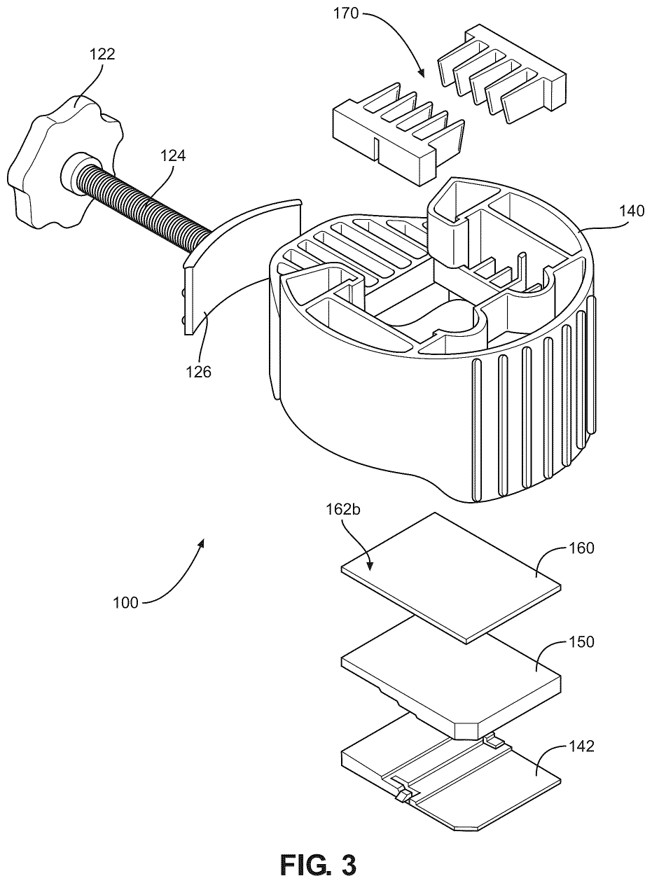

FIG. 1 is a front perspective view of a quality assurance phantom 100 for digital radiography according to an embodiment of the present general inventive concept, FIG. 2 is bottom perspective view of the quality assurance phantom 100 for digital radiography illustrated in FIG. 1, and FIG. 3 is an exploded assembly view of the quality assurance phantom 100 illustrated in FIG. 1, with housing 110 removed.

Referring to FIGS. 1-3, according to an embodiment of the present general inventive concept, the quality assurance phantom (designated generally as 100) includes a housing 110 having a first side 110a and an opposing second side 110b and an adjustable clamp member 120 configured to detachably couple the housing 110 to various types of digital imaging systems.

In the present embodiment, the housing 110 includes an alignment frame 140 designed and configured to align a contrast layer 150 and a spatial resolution test pattern layer 160 to a central axis A1 of the housing 110. The housing 110 further includes at least one sidewall 116 that helps align the housing 110 to a transmission axis A3 of conventional digital imaging systems.

Referring to FIG. 3, in the present embodiment, the alignment frame 140 is designed and/or configured to receive and align the contrast layer 150 and the spatial resolution layer 160 to a central axis A2 of the alignment frame 140. The alignment frame 140 further includes an alignment frame cap 142 that is configured to secure the contrast layer 150 and the spatial layer 160 to the alignment frame 140. The contrast layer 150 is designed to assess a contrast resolution or contrast visibility of the x-ray device and the spatial resolution layer 160 is designed to assess a spatial resolution of the x-ray device.

Contrast visibility refers to the ability of the x-ray device to distinguish between different levels of contrast in terms of gray scale of the contrast layer 150.

In the present embodiment, the contrast layer 150 is formed from a single material having one or more sections, each section having a different thickness. The single material includes one of porcelain, aluminum, PTFE, POM, and PMMA. However, the contrast layer materials are not limited thereto.

In alternative embodiments, the contrast layer 150 is formed from one or more different materials, wherein the contrast layer has a uniform thickness and made of porcelain, aluminum, PTFE, POM, and PMMA. However, the present general inventive concept is not limited thereto.

In the present embodiment, the spatial resolution layer 160 includes one or more traces 164 disposed on the top surface (i.e. a first surface) of a substrate 162. The traces 164 are formed of copper with different thicknesses and are spaced apart at different widths. However, the present general inventive concept is not limited thereto.

In the present embodiment, the substrate 162 may be formed using a PCB material. However, the present general inventive concept is not limited thereto. That is, in alternative embodiments, the substrate 162 may be formed of various non-conductive materials.

In the present embodiment, the adjustable clamp member 120 includes a rotatable handle 122 attached to a threaded portion 124 that is coupled to a contact pad 126. The threaded portion 124 passes through a sidewall 116 of the housing 110. As such, a rotation of the handle 122 in a first direction (i.e., a clockwise direction) moves the contact pad 126 away from the handle 122 in order to tighten against an outer surface of a conventional digital imaging system. As a result, the quality assurance phantom 100 will self-align with an x ray transmission axis of the digital imaging system.

Conversely, a rotation of the handle 122 in a second direction (i.e., a counter-clockwise direction) moves the contact pad 126 toward the handle 122 to be released/removed from an outer surface of a conventional digital imaging system. In other words, the handle 122 of the adjustable clamp member 120 is designed to allow a user to quickly align, attach and detach the housing 110 from a digital imaging system.

In alternative embodiments, the contact pad 126 may be formed in various shapes and sizes as desired. In the present embodiment, the contact pad 12 is formed in a curved shape so as to correspond or match a curve of an exterior surface of the x-ray device 10. However, the present general inventive concept is not limited thereto.

In alternative embodiments, the quality assurance phantom 100 further includes a release mechanism 130 that is movable from a first position P1 to a second position P2 and designed to eject/push out a digital sensor 12 installed within the opening 112 of the housing 110.

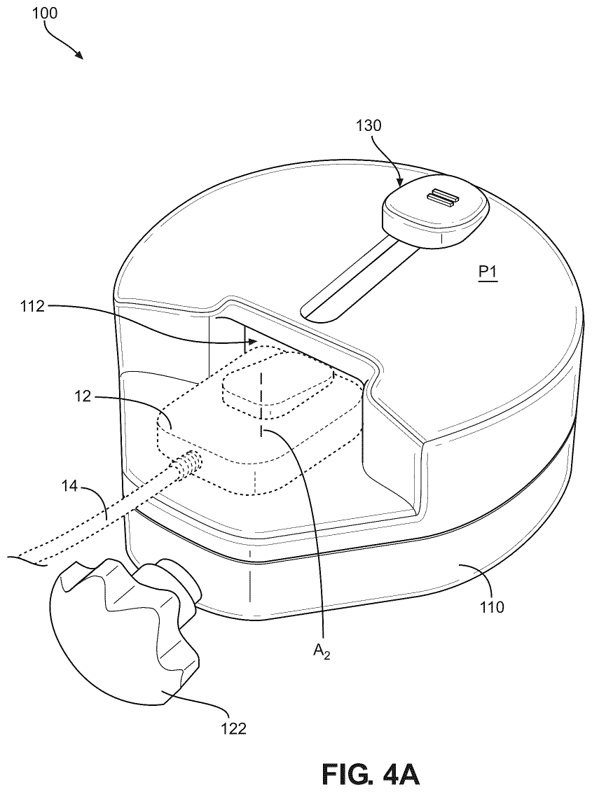

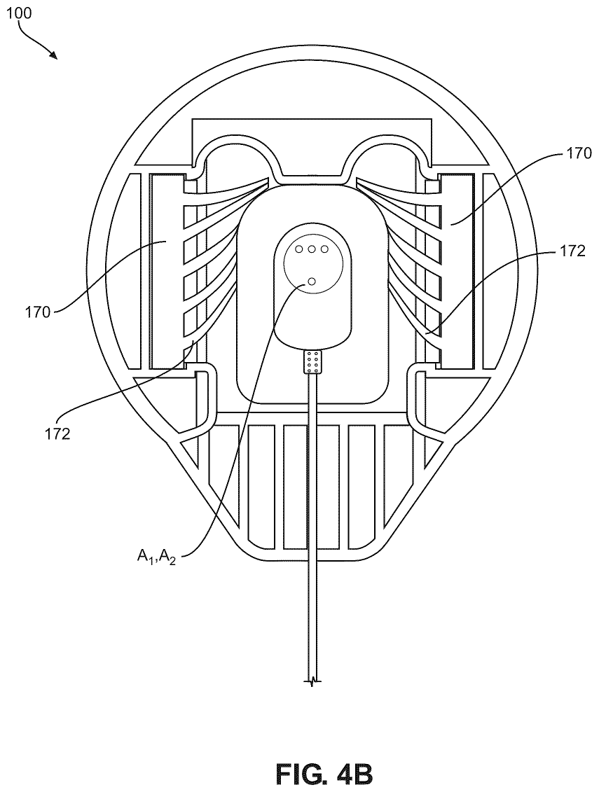

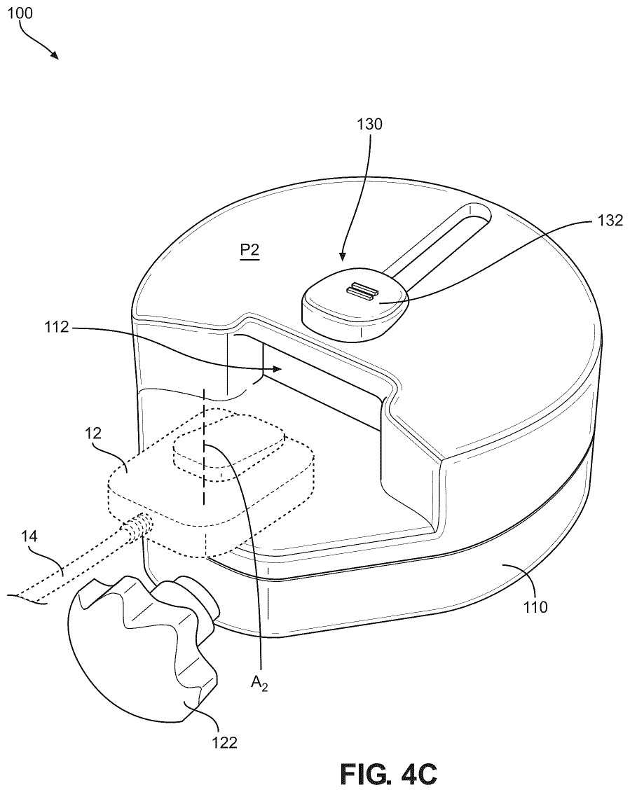

FIG. 4A is a front perspective assembly view of a conventional digital imaging sensor 12 partially assembled into the quality assurance phantom 100 illustrated in FIG. 1, FIG. 4B is a top assembly view of a conventional digital imaging sensor 12 completely assembled into the quality assurance phantom 100 illustrated in FIG. 1, with housing 110 removed, and FIG. 4C is a front perspective assembly view of a conventional digital imaging sensor 12 partially ejected using a release mechanism 130.

Referring to FIG. 4A, in the present embodiment, the quality assurance phantom 100 further includes an opening 112 configured to receive a conventional digital sensor 12 that is used with conventional digital imaging systems. The opening 112 is designed to receive various sized digital sensors, including sizes 0, 1, 1.5, and 2. However, the present general inventive concept is not limited thereto.

Referring to FIG. 4B, the quality assurance phantom 100 further includes a flexible holder 170 that is coupled to the alignment frame 140 on opposing sides of the opening 112 and adjacent to a bottom surface 162b (i.e. a second surface) of the substrate 162. The alignment frame 140 is designed and/or configured to align the digital sensor 12 with a central axis A1 of the alignment frame 140. As such, as the digital sensor 12 is inserted within the housing 110, the flexible holder 170 self-aligns a central axis A2 of the digital sensor 12 with the central axis A1 of the housing 110 and the alignment frame 140. The alignment frame 140 further includes a sensor position stop 142 that is designed to control a position/location of a digital sensor inserted within the housing 110.

In alternative embodiments, the flexible holder 170 is formed of a flexible or semi-flexible material such as plastic or silicone to dynamically secure various sized digital sensors 12. The flexible holder 170 may further include a plurality of flexible members or teeth 172 that are configured to further secure and align the digital sensor 12 with respect to the frame member 140. That is, the flexible members 172 of the flexible holder 170 allow a user to quickly and easily align and secure digital sensors 12 of sizes 0 to 2 into the alignment frame 140 of the quality assurance phantom 100.

Referring to FIG. 4C, the release mechanism 130 may further include a slidable button 132 designed to operate the release mechanism 130 using a single finger to eject/push out the digital sensor 12 from the housing 110 and the alignment frame 140 in order to prevent damage to the digital sensor 12 and cable 14 of the digital sensor.

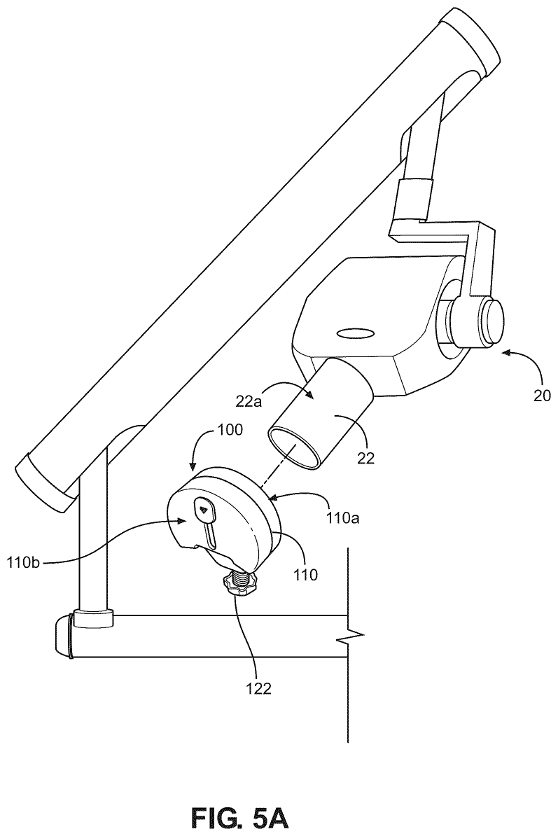

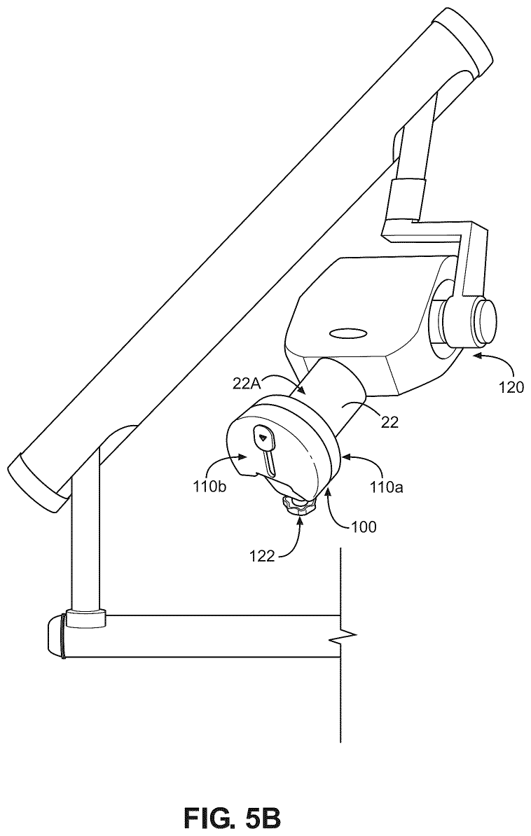

FIG. 5A is an exploded assembly view of a quality assurance phantom 100 according to an embodiment of the present general inventive concept and a conventional dental x-ray device 20 and FIG. 5B is a completed assembly view of the quality assurance phantom 100 and the conventional dental x-ray device 20 illustrated in FIG. 5A.

In operation, referring to FIGS. 5A and 5B, the quality assurance phantom 100 is detachably coupled to a dental x-ray device 20. That is, in the present embodiment, the first side 110a of the housing 110 is aligned with a beam limiting device 22 of the x-ray device 20. Next, the handle 122 of the adjustable clamp member 120 is rotated in the first direction (i.e., a clockwise direction) in order to move the contact pad 126 toward an outer surface 22a of the beam limiting device 22. As such, when the contact pad 126 makes contact with the outer surface 22a of the beam limiting device 22, the housing 100 self-centers with the beam limiting device 22. Once the clamp member 120 is completely tightened, the quality assurance phantom 100 is centered with respect to the beam limiting device 22 of the x-ray device 20.

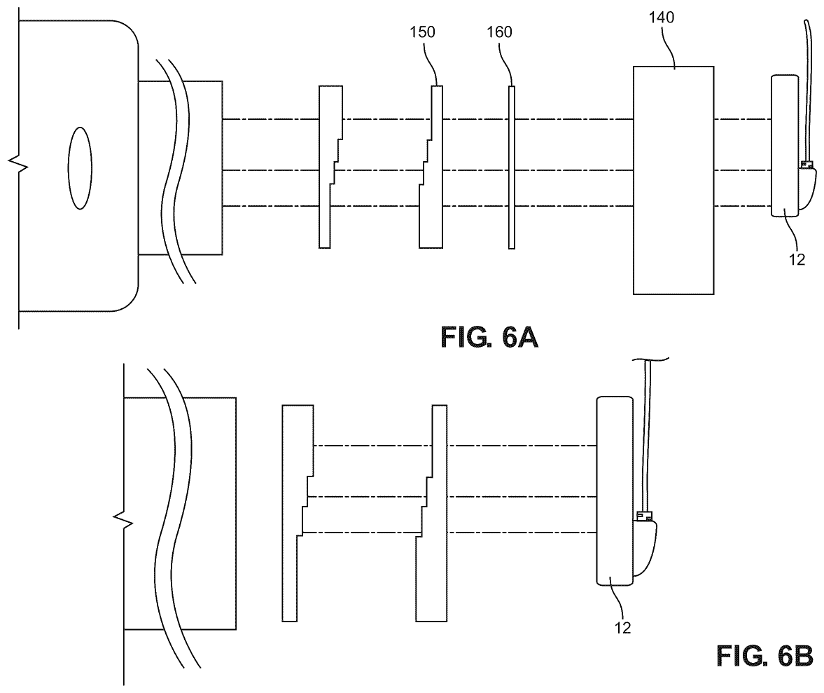

FIG. 6A is a schematic illustration of an alignment of a contrast layer, a spatial resolution layer, and alignment frame of the quality assurance phantom and a digital sensor with an axis of a conventional dental x-ray device and FIG. 6B is an enlarged schematic illustration of FIG. 6A, with the alignment frame removed.

Referring to FIGS. 6A and 6B, the quality assurance phantom 100 according to the present inventive concept utilizes the alignment frame 130 to precisely and repeatably align the contrast layer 150 with the spatial resolution layer 160 on one side of the alignment frame 130 and precisely and repeatable align the digital sensor 12 on the other side of the alignment frame 130. As such, when the housing 110 is aligned and secured with the x ray transmission axis of the digital imaging system, the alignment frame 130 and in turn, the contrast layer, the spatial resolution layer 160, and the digital sensor are also aligned with the x-ray transmission axis of the digital imaging system.

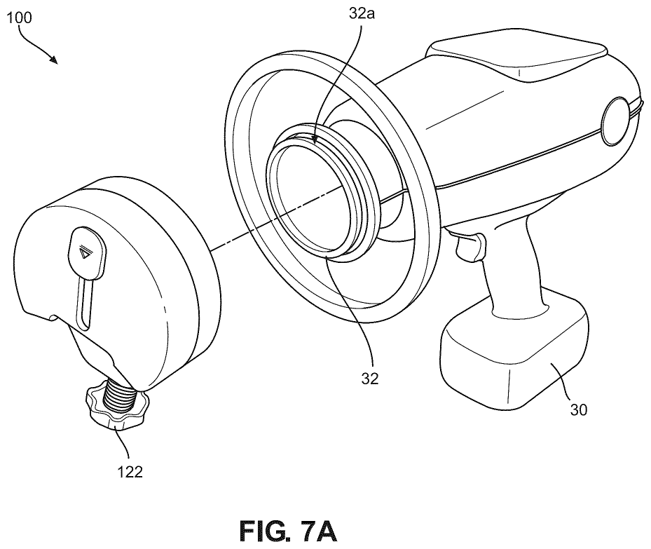

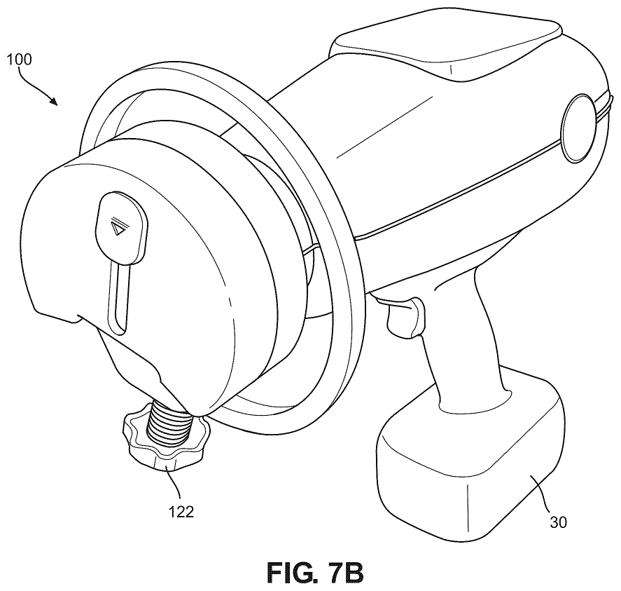

FIG. 7A is an exploded assembly view of a quality assurance phantom 100 according to an embodiment of the present general inventive concept and a conventional hand-held dental x-ray device 30 and FIG. 7B is an assembly view of the quality assurance phantom 100 and the conventional hand-held dental x-ray device 30 illustrated in FIG. 7A.

In operation, referring to FIGS. 7A and 7B, the quality assurance phantom 100 is detachably coupled to a hand-held dental x-ray device 30. That is, in the present embodiment, the first side 110a of the housing 110 is aligned with a beam limiting device 32 of the hand-held dental x-ray device 30. Next, the handle 122 of the adjustable clamp member 120 is rotated in the first direction (i.e., a clockwise direction) in order to move the contact pad 126 toward an outer surface 22a of the beam limiting device 22. As such, when the contact pad 126 makes contact with the outer surface 32a of the beam limiting device 32, the housing 110 self-centers with the beam limiting device 32. Once the clamp member 120 is completely tightened, the quality assurance phantom 100 is centered with respect to the beam limiting device 32 of the hand-held dental x-ray device 30.

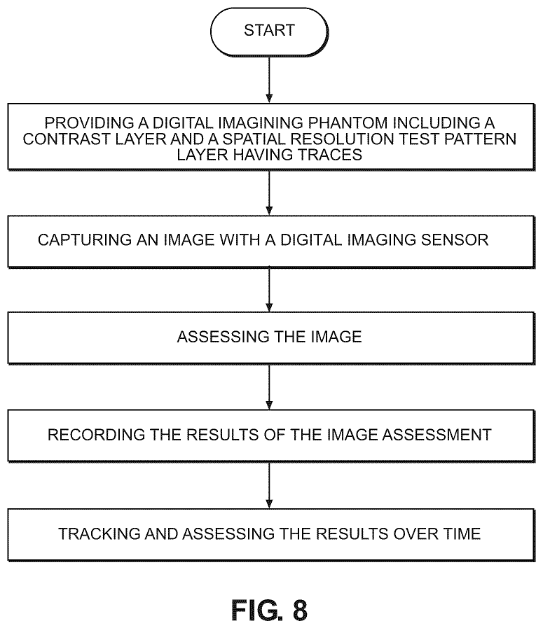

FIG. 8 is a flowchart of a method of assuring the quality of digital imaging system according to an embodiment of the present inventive concept.

Referring to FIG. 8, in an alternative exemplary embodiment, the present general inventive concept provides a method of assuring the quality of digital imaging 200. The exemplary method 200 includes a digital imaging system having an imaging acquisition sensor and an x-ray source, The exemplary method 200 comprises a step 202 of providing a digital imagining phantom including a contrast layer including different contrast levels and that is configured to assess the contrast resolution of the digital imaging system. The digital imaging phantom further includes a printed circuit board spatial resolution test pattern layer that includes a printed circuit board having copper traces of variable pattern and thickness. The spatial resolution test layer is configured to assess the spatial resolution of the digital imaging system. The exemplary method 200 further includes a step 204 of creating a baseline image, including capturing an image with an imaging acquisition sensor. The exemplary method 200 further includes a step 206 of assessing the image for contrast, spatial resolution, artifacts, and noise using the contrast layer and the printed circuit board spatial resolution test pattern layer. The exemplary method 200 further includes a step 206 of recording the results of the image assessment. The exemplary method 200 further includes a step 208 of tracking and assessing the results over time. However, the present general inventive concept is not limited thereto.

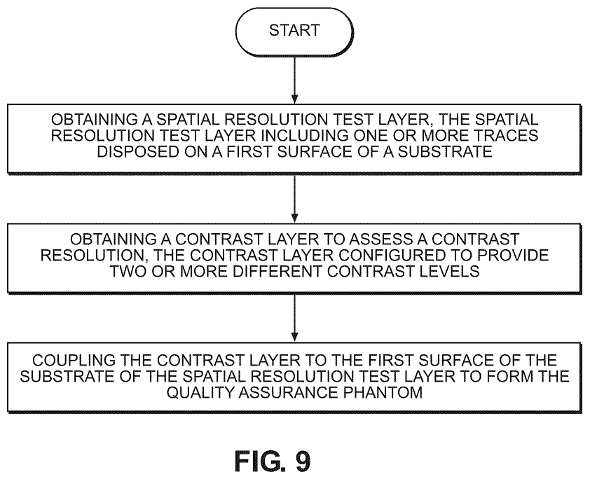

FIG. 9 is a flowchart of a method of creating a quality assurance phantom for a x-ray device according to an embodiment of the present inventive concept.

Referring to FIG. 9, in an alternative exemplary embodiment, the present general inventive concept provides a method of creating a quality assurance phantom for an x-ray device 300. The exemplary method 300 includes a step 302 of obtaining a spatial resolution test layer, the spatial resolution test layer including one or more traces disposed on a first surface of a substrate. The exemplary method 300 further includes a step 304 of obtaining a contrast layer to assess a contrast resolution, the contrast layer configured to provide two or more different contrast levels. The exemplary method 300 further includes a step 306 of coupling the contrast layer to the first surface of the substrate of the spatial resolution test layer to form the quality assurance phantom. However, the present general inventive concept is not limited thereto.

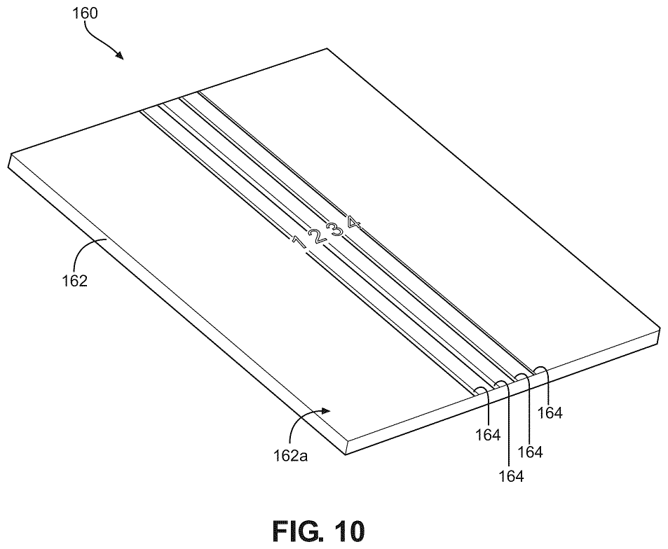

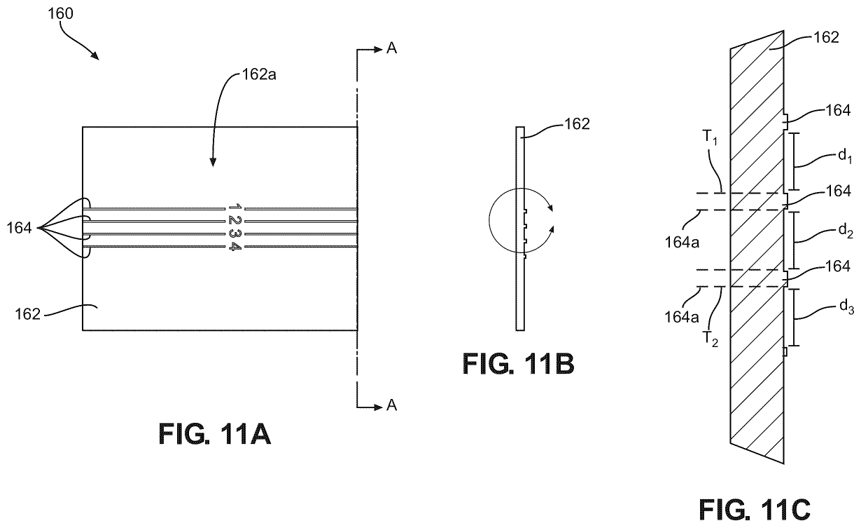

FIG. 10 is a front perspective view of the spatial resolution test pattern layer of the quality assurance phantom illustrated in FIG. 3, FIG. 11A is a top plan view of the spatial resolution test pattern layer illustrated in FIG. 10, and FIG. 11B is a cross-sectional view of the spatial resolution test pattern layer in FIG. 11A, along the A-A line. FIG. 11C is an enlarged schematic view of the spatial resolution test pattern layer illustrated in FIG. 11B.

Referring to FIGS. 10 and 11A-C, in the present general inventive, the spatial resolution layer 160 comprises a substrate 162 with at least one trace 164 disposed on a first surface 162a of substrate 162. The spatial resolution test pattern layer 130 is comprised of a substrate 132 includes a plurality of traces 164. The exemplary substrate 162 may be formed with a nonconductive material. However, the present general inventive concept is not limited thereto. The exemplary substrate 132 may be formed with silicon.

The exemplary traces 164 are formed such that each of the plurality of traces 164 has a different thickness 164a. The exemplary traces 164 are arranged on the substrate 162 such that each trace 164 is spaced apart from the immediately adjacent traces 164 a specific width D1, and each width between the traces 164 may be different. The exemplary traces 164 may be comprised of a conductive material, such as copper or a copper material. However, the present general inventive concept is not limited thereto.

In alternative exemplary embodiments, the exemplary traces 164 may be arranged on the substrate 162 in a fan pattern in which a first end of the traces 164 are arranged in close proximity to one another, and the traces 164 are spaced further apart at a second end of the traces 164. In other alternative embodiments, the traces 164 may be arranged on substrate 162 in different patterns or shapes. However, the trace arrangements are not limited thereto.

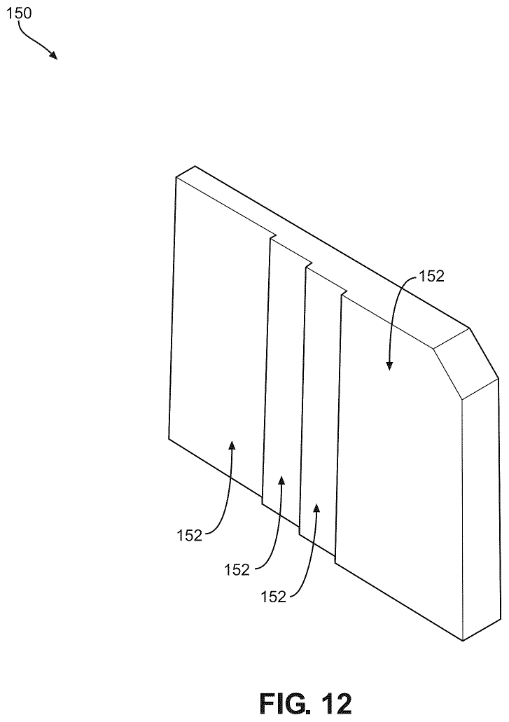

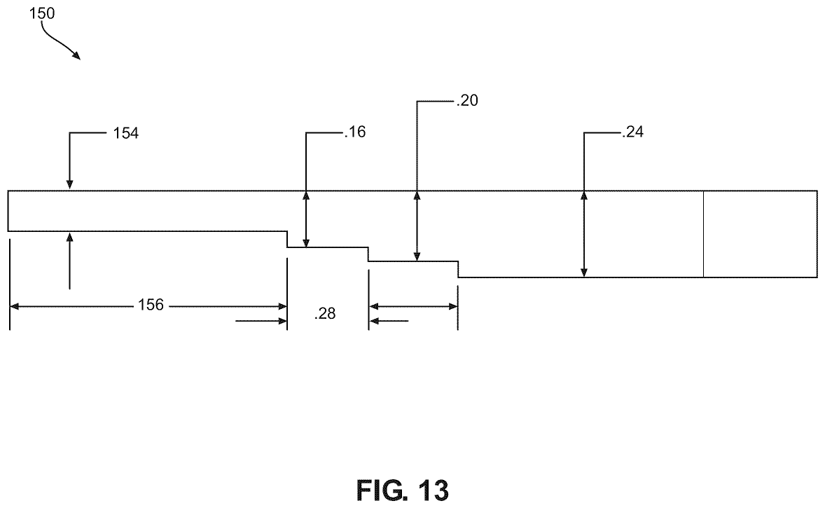

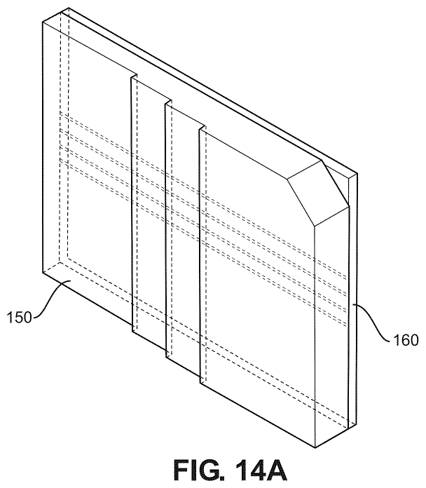

FIG. 12 is a front perspective view of a contrast layer 150 of the quality assurance phantom illustrated 100 in FIG. 3 and FIG. 13 is a side view of the contrast layer 150 of the quality assurance phantom 100 illustrated in FIG. 12.

Referring to FIGS. 12 and 13, in the present embodiment, the contrast layer 150 configured to assess a contrast resolution. The exemplary contrast layer 150 is configured to provide two or more different contrast layers. The exemplary contrast layer 150 comprises a material sufficient to assess contrast resolution.

In the present embodiment, the contrast layer 150 comprises an aluminum sheet having a plurality of steps 152. Each step 152 is configured to have a different depth 154 and width 156, thereby providing different contrast levels. For example, FIG. 13 shows exemplary depths 154 and widths 156 of exemplary steps 152. However, the present general inventive concept is not limited thereto.

In alternative exemplary embodiments, the contrast layer 150 may comprise a sheet of a plurality of materials each having different contrast properties. Alternative contrast layers 150 may be configured to create different contrast levels to mimic different tissues or other anatomical structures. For example, in alternative embodiments, the contrast layer 150 may be comprised of at least one of the following materials: porcelain; PTFE; POM; and PMMA. However, the contrast layer materials are not limited thereto.

In alternative exemplary embodiments, the exemplary housing 110, contrast layer 150, and spatial resolution layer 160 may be manufactured to be larger or smaller to accommodate other applications other than dentistry, for example, extra-oral applications, chiropractic applications, veterinarian applications, medical applications, or industrial applications. However, the potential applications are not limited hereto.

In alternative exemplary embodiments, for example, in computerized radiography or chemical radiography, the housing 110 may further include an opening and space for films or intra-oral films adjacent the flexible holder 170. For example, the film may be slid into an opening immediately below the flexible teeth 172. However, the present general inventive concept is not limited thereto.

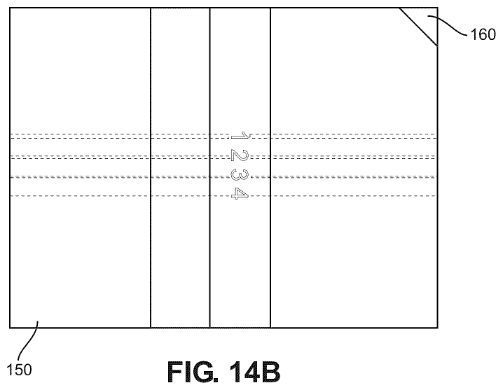

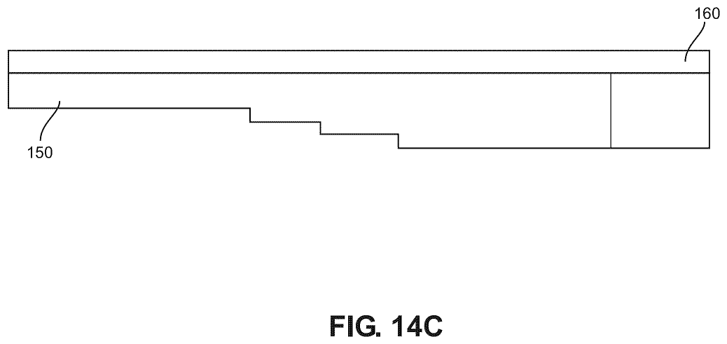

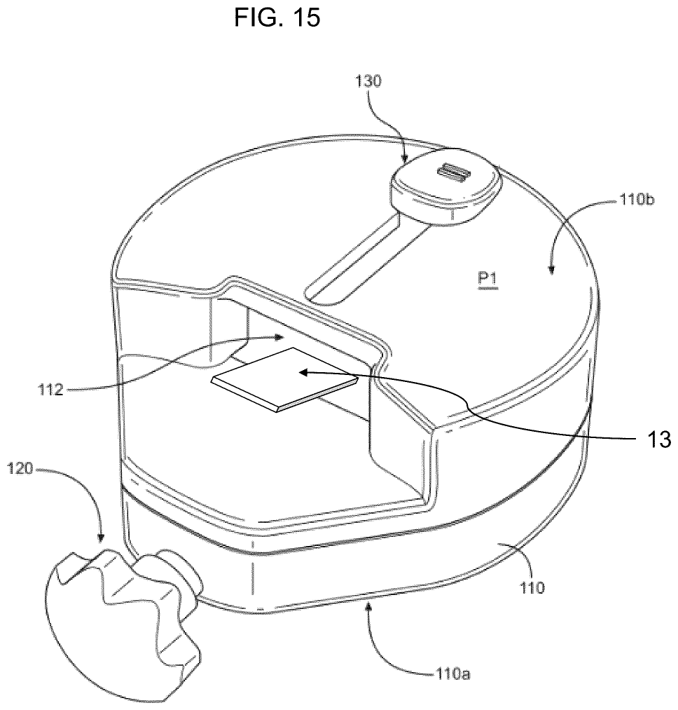

FIG. 14A is a front perspective assembly view of a contrast layer coupled to a spatial resolution test pattern layer with hidden lines visible illustrating traces disposed on the substrate, according to an embodiment of the present invention, FIG. 14B is a top plan view of the contrast layer coupled to the spatial resolution test pattern layer illustrated in FIG. 14A and FIG. 14C is a side view of the contrast layer coupled to the spatial resolution test pattern layer illustrated in FIG. 14A.

In exemplary embodiments, the exemplary contrast layer 150 is arranged and aligned in the quality assurance phantom 100 such that contrast layer 150 is overlayed on top of the spatial resolution layer 160. In alternative exemplary arrangements, the exemplary contrast layer 150 may be arranged in the housing 110 such that the spatial resolution layer 160 is overlayed directly over the contrast layer 150. In alternative exemplary embodiments, two or more contrast layers 150 and PCB spatial resolution layers 160 may be formed within the housing 110. However, the present general inventive concept is not limited thereto.

In operation, a digital sensor 12 (e.g., a digital intra-oral sensor) is inserted into the opening 112 of housing 110. The flexible holder 170 self-aligns the digital sensor 12 to the opening 112 and dynamically secures the digital sensor 12 to the housing 110. That is, the flexible holder 170 is designed to bend in such a manner so as to maintain an alignment of the digital sensor 12 to the opening 112 during insertion.

Next, a user would execute a quality assurance application that implements a method according to the present invention of assuring the quality of digital imaging of various types of x-ray systems over time. That is, the present general inventive concept further includes software (or application) that enables a user to record, track, assess, and maintain a digital image quality assurance program for radiography.

The user would use the quality assurance application to select a previously entered quality assurance protocol for digital images for the particular digital sensor being tested. Once selected, the quality assurance application retrieves the selected quality assurance (QA) protocol from a data storage and displays the selected protocol instructions and x-ray technique settings to be used for the image acquisition on a display screen. The quality assurance application further instructs the user to set the x-ray device 20 settings of kV, mA, and time(s) and then initiate the x-ray device 20.

Once the x-ray device 20 emits the x-ray, an x-ray image is displayed on the user's dental software and the QA application instructs the user to assess the image for quality assurance purposes. That is, the QA application instructs the user to observe the image and record on an electronic QA report form how many shades of gray (i.e., contrast levels) appear in the image, how many lines (i.e., spatial resolution) appear in the darkest region of the image (i.e., high contrast spatial resolution), and how many lines appear in the lightest region of the image (i.e., low contrast spatial resolution). In addition, the QA application further instructs the user to record any additional information on observed artifacts, such as bite marks. The QA application then instructs the user to digitally sign the QA report.

In another embodiment of the invention, the user is asked to export the image from their dental software and upload it to the QA application according to the present invention.

In yet another embodiment, the QA application according to the present invention automatically captures the image by communicating with the digital sensor. Once the image is captured by the QA application, an algorithm assesses the image and automatically computes the aforementioned contrast levels, spatial resolution, artifacts, and noise metrics without human intervention. The QA application maintains a log of all QA reports and data, as well as tracks the image quality over time to alert the end-user of image quality degradation (if any). All reports logs are available to be inspected by the end-user or a state compliance inspector to maintain compliance with state regulations and standards for digital imaging.

After the x-ray image is captured and analyzed by the software, the end-user can easily access and review the QA report form and image quality metrics. The software also keeps a record of all QA reports and data, which can be accessed by the end-user or state compliance inspector to ensure compliance with state regulations and standards for digital imaging for x-ray machines.

In addition, the device is designed for ease of use and improved usability. The flexi pocket holder allows the end-user to quickly insert and detach digital sensors without using clamps, reducing the time of test and improving usability. The plastic housing contains a push button for quick removal of the digital intra-oral sensors, preventing damage to the sensor cable or sensor. The side screw gripping mechanism with handle enables the user to quickly attach and detach the housing from the x-ray machine.

Overall, the present invention provides an innovative and novel method of utilizing manufacturing processes for PCBs, achieving similar spatial resolution patterns or more complex design patterns using copper traces on PCBs. This allows for precise control of copper thickness and width, as well as cheap production at scale. By encapsulating the contrast and spatial resolution layers into a single housing designed to attach directly to the x-ray machine, the present invention improves quality, speed of testing, and repeatability by being able to consistently position and align the quality assurance phantom with respect to the digital sensor and the x-ray source.

Further, the present invention provides a method of using an application to capture the x-ray generated image of the quality assurance phantom, and then using an algorithm to automatically assess the image and compute the aforementioned contrast levels, spatial resolution, artifacts, and noise metrics, without human intervention. The software according to present invention also maintains a log of all QA reports and data, as well as tracks the image quality over time to alert the user of any potential image quality degradation.

Furthermore, all reports and logs are available for inspection by the end-user or state compliance inspector to ensure compliance with state regulations and standards for digital imaging for x-ray machines. This quality assurance phantom and application (i.e., quality assurance system) according to the present invention allows for efficient and reliable quality assurance testing of digital intra-oral sensors, as well as improved repeatability and accuracy in testing.

This quality assurance phantom and application (i.e., quality assurance system) also includes a feature for automated image analysis, where the application automatically analyzes and reports on the images, reducing the time and effort required by the end-user in analyzing and interpreting the images.

FIG. 15 is a front perspective view of a quality assurance phantom for digital radiography according to an alternative embodiment of the present general inventive concept.

Referring now to FIG. 15, in alternative embodiments, the digital sensor 12 may be foregone and a phosphorus plate 13 may be used instead. The exemplary phosphorus plate 13 comprises a flexible, reusable imaging plate coated with a phosphorus layer. In alternative embodiments, any type of phosphorus plate known in radiography may be used. The exemplary phosphorus plate 13 is configured to be installed within the opening 112 of the housing 110 in a similar as digital sensor 12. However, the present general inventive concept is not limited thereto.

Although a few exemplary embodiments of the present general inventive concept have been illustrated and described, it will be appreciated by those skilled in the art that changes may be made in these exemplary embodiments and other embodiments may be made from the various features and relationships without departing from the principles and spirit of the general inventive concept, the scope of which is defined in the appended claims and their equivalents.

What is claimed is:

1. A quality assurance phantom configured for connection to an x-ray device, comprising:
a housing member having an opening configured to receive a digital sensor or phosphorus plate;
a frame member disposed within the housing member, the frame member configured to receive a phantom device, the phantom device comprising;
a contrast layer configured to assess a contrast resolution, wherein the contrast layer is configured to provide two or more different contrast levels; and
a spatial resolution test layer, wherein the spatial resolution test layer includes at least one trace disposed on a first surface of a substrate; and
an attachment member configured to detachably secure the housing member to the x-ray device.

2. The quality assurance phantom of claim 1, further comprising a flexible holder coupled to the frame member and configured to align a central axis of the digital sensor or phosphorus plate with a central axis of the frame member.

3. The quality assurance phantom of claim 2, wherein the flexible holder further includes a plurality of flexible members configured to secure and align the digital sensor or phosphorus plate with respect to the frame member.

4. The quality assurance phantom of claim 3, further comprising a release mechanism movable from a first position to a second position and configured to eject the digital sensor or phosphorus plate from the opening of the housing member.

5. The quality assurance phantom of claim 1, wherein the attachment member includes an adjustable clamp member configured to align an x-ray transmission axis of the x-ray device with the phantom device.

6. The quality assurance phantom of claim 5, wherein the clamp member includes a curved contact pad configured to contact an exterior surface of the x-ray device.

7. The quality assurance phantom of claim 6, wherein the contrast layer is formed from a single material having one or more sections, each section having a different thickness.

8. The quality assurance phantom of claim 7, wherein the material includes one of porcelain, aluminum, PTFE, POM, and PMMA.

9. The quality assurance phantom of claim 6, wherein the contrast layer is formed from one or more different materials, the contrast layer having a uniform thickness.

10. The quality assurance phantom of claim 9, wherein the material includes one of porcelain, aluminum, PTFE, POM, and PMMA.

11. The quality assurance phantom of claim 1, wherein the substrate includes a PCB material.

12. The quality assurance phantom of claim 1, wherein the one or more traces are formed with different thicknesses.

13. The quality assurance phantom of claim 12, wherein adjacent traces are spaced apart at different widths.

14. The quality assurance phantom of claim 13, wherein the traces are formed with a copper material.

15. The quality assurance phantom of claim 1, wherein the substrate includes a nonconductive material.

16. A method of creating a quality assurance phantom for an x-ray device, the method comprising:
obtaining a spatial resolution test layer, the spatial resolution test layer including one or more traces disposed on a first surface of a substrate;
obtaining a contrast layer to assess a contrast resolution, the contrast layer configured to provide two or more different contrast levels; and
coupling the contrast layer to the first surface of the substrate of the spatial resolution test layer to form the quality assurance phantom.

17. The method of claim 16, wherein the one or more traces are formed with different thicknesses.

18. The method of claim 16, wherein the one or more traces are spaced apart at different widths.

19. The method of claim 17, wherein the substrate includes a nonconductive material.

20. The method of claim 16, wherein the traces are formed with a conductive material.

21. The method of claim 20, wherein the traces are formed with a copper material.

22. The method of claim 16, wherein the contrast layer is formed from a single material having one or more sections, each section having different thicknesses.

23. The method of claim 22, wherein the material includes one of porcelain, aluminum, PTFE, POM, and PMMA.

24. The method of claim 16, wherein the contrast layer is formed from one or more different materials, the contrast layer having a uniform thickness.

25. A method of assuring the quality of digital imaging including a digital imaging system having a digital imaging sensor or phosphorus plate and an x-ray source, the method comprising the steps of:
providing a digital imagining phantom comprising:
a contrast layer, wherein the contrast layer is comprised of different contrast levels and is configured to assess the contrast resolution of the digital imaging system;
a spatial resolution test pattern layer, wherein the spatial resolution test pattern layer includes copper tracings of variable pattern and thickness, wherein the spatial resolution test layer is configured to assess the spatial resolution of the digital imaging system;
creating a baseline image, the creating step comprising:
capturing an image with at least one of the digital imaging sensor and the phosphorus plate;
assessing the image for contrast, spatial resolution, artifacts, and noise using the contrast layer and the printed circuit board spatial resolution test pattern layer;
recording the results of the image assessment; and
tracking and assessing the results over time.
